(19) United States
(12) Patent Application Publication
(10) Pub. No.: US 2026/0364278 A1
(43) Pub. Date: Nov. 19, 2026

(54) NEBULIZER

(71) Applicant: Chilone Inc., Denver, CO (US)

(72) Inventor: Yongqing WU, Denver, CO (US)

(21) Appl. No.: 19/208,577

(22) Filed: May 14, 2025

(51) Int. Cl. A61M 11/02; A61M 11/00; A61M 16/06

(52) U.S. Cl. CPC A61M 11/02; A61M 11/005; A61M 16/0683; A61M 2205/18; A61M 2205/3386; A61M 2205/502; A61M 2205/505; A61M 2205/581; A61M 2205/583

(57) ABSTRACT

a nebulizer, the nebulizer is a portable handheld device, including a housing, an atomizing component, a controller and a display. The atomizing component is disposed in the housing, and the atomizing component is configured to atomize liquid in a liquid storage tank. The controller is disposed in the housing, the controller is electrically connected to the atomizing component. The display device includes a display screen, the display screen is disposed in the housing and at least partially exposed from the housing, the display screen is electrically connected to the controller, and the display screen is configured to display parameter information of the nebulizer. The nebulizer is easy to control and is convenient to use.

### TECHNICAL FIELD

The present disclosure relates to the technical field of nebulizer technology, in particular to a nebulizer.

### BACKGROUND

At present, nebulizers on the market are prompted by lights (such as color or flashing) to indicate the mode, and users cannot intuitively understand the device situation. When using colors to indicate information, the difficulty of understanding the device's condition will greatly increase for color blind or older users, seriously affecting their user experience.

### SUMMARY

The present disclosure provides a nebulizer, to help users to understand the situation of the nebulizer.

To realize the above objective, the present disclosure provides a nebulizer, the nebulizer is a portable handheld device, including a housing, an atomizing component, a controller and a display; the atomizing component is disposed in the housing, and the atomizing component is configured to atomize liquid in a liquid storage tank; the controller is disposed in the housing, the controller is electrically connected to the atomizing component; the display device includes a display screen, the display screen is disposed in the housing and at least partially exposed from the housing, the display screen is electrically connected to the controller, and the display screen is configured to display parameter information of the nebulizer.

Furthermore, the display screen is a light emitting diode display screen or a liquid crystal display screen.

Furthermore, the display screen is integrated with a touching member, and the display screen is touch controlled.

Furthermore, the nebulizer further comprises a control button, the control button is disposed in the housing and electrically connected to the controller.

Furthermore, the display screen is equipped with an adaptive adjusting device; when the nebulizer is in an inclined use state, the adaptive adjusting device is configured to switch the display screen from a portrait mode layout to a landscape mode layout.

Furthermore, the nebulizer further comprises a liquid level sensor and a warning device; the liquid level sensor is configured to detect the liquid level; when the liquid level sensor detects that the liquid level is below a first threshold, the warning device is configured to trigger the display screen to flash an icon or emit warning sound.

Furthermore, the nebulizer further comprises a face mask and a reflux assembly; the face mask is connected to the housing and communicated with the atomizing component; the reflux assembly is connected to the face mask and the liquid storage tank of the atomizing component, the reflux assembly is configured to guide the liquid condensed in the face mask back to the liquid storage tank.

Furthermore, an inner wall of the face mask is provided with a recovery groove, the recovery groove is configured to hold the condensed liquid; the reflux assembly comprise a reflux pump and a conduit, the conduit is connected to the recovery groove and the reflux pump.

Furthermore, the nebulizer further comprises a face mask and a strap; the face mask is connected to the housing and communicated with the atomizing component; two ends of the strap are connected to the face mask, and the strap is configured to be worn over a head of a user; when the strap is worn over the head of the user, the face mask is configured to support the housing.

Furthermore, a surface of the housing is provided with a protruding part, the protruding part is extended in a direction away from the surface of the housing, the face mask is worn over the protruding part.

Furthermore, the surface of the housing is provided with an annular concave part, the face mask is inserted into the annular concave part.

Furthermore, the atomizing component comprises a liquid storage tank, the liquid storage tank is exposed from the housing, and at least a portion of the liquid storage tank is made of a transparent material.

Furthermore, the atomizing component comprises a liquid storage tank, the liquid storage tank is exposed from the housing; the liquid storage tank comprises a tank body and a tank cover, the tank cover is rotatably connected to the tank body.

Furthermore, one of the tank body and the tank cover is provided with a connecting part, and the other of the tank body and the tank cover is provided with a latching part, the latching part is detachably and elastically snap-connected to the connecting part.

Furthermore, the atomizing component comprises a liquid storage tank, the liquid storage tank is exposed from the housing, and is detachably connected to the housing.

The present disclosure further provides a nebulizer, the nebulizer is a portable handheld device, comprising a housing, an atomizing component, a controller and a display. The atomizing component is disposed in the housing, and the atomizing component is configured to atomize liquid in a liquid storage tank. The controller is disposed in the housing, the controller is electrically connected to the atomizing component. The display device includes a display screen, the display screen is disposed in the housing and at least partially exposed from the housing, the display screen is electrically connected to the controller, and the display screen is configured to display parameter information of the nebulizer. It is easy for users to understand the situation of the nebulizer. The nebulizer is easy to control and is convenient to use.

### BRIEF DESCRIPTION OF THE DRAWINGS

FIG. 1 is a schematic structural diagram of a nebulizer according to an embodiment of the present disclosure.

FIG. 2 a schematic structural diagram of a part of the nebulizer according to an embodiment of the present disclosure.

FIG. 3 is a schematic structural diagram of the nebulizer in FIG. 2 with a tank cover partially opened.

FIG. 4 is a schematic structural diagram of the nebulizer in FIG. 2 with the tank cover fully opened.

FIG. 5 is an exploded diagram of the nebulizer in FIG. 1.

FIG. 6 is a schematic structural diagram of the nebulizer according to another embodiment of the present disclosure.

FIG. 7 is an exploded diagram of the nebulizer in FIG. 6.

FIG. 8 is a schematic structural diagram of the nebulizer according to another embodiment of the present disclosure.

FIG. 9 is a schematic structural diagram of a part of the nebulizer according to another embodiment of the present disclosure.

### DESCRIPTION OF THE REFERENCE NUMERAL

nebulizer 100, housing 10, protruding part 11, annular concave part 12, atomizing component 20, liquid storage tank 21, tank body 211, opening 2111, connecting part 2112, tank cover 212, latching part 2121, groove 2122, controller 30, circuit board 31, display device 40, display screen 41, touching member 42, adaptive adjusting device 43, control button 51, liquid level sensor 52, warning device 53, face mask 61, recovery groove 611, strap 62, reflux assembly 63, conduit 631, reflux pump 632, battery 71, charging interface 72.

The following embodiments will be further explained in conjunction with the attached drawings.

### DETAILED DESCRIPTION OF THE EMBODIMENTS

The technical solutions in the embodiments of the present disclosure will be described clearly and completely with reference to the accompanying drawings in the embodiments of the present disclosure. Obviously, the described embodiments are only a part of the embodiments of the present disclosure rather than all of them. Based on the embodiments of the present disclosure, all other embodiments obtained by those skilled in the art without creative work shall fall within the scope of protection of the present disclosure.

It should be noted that when a component is considered to be “connected” to another component, it can either be directly connected to the other component or may have an intermediate component positioned between them. When a component is considered to be “arranged” on another component, it can either be directly placed on another component or may have an intermediate component positioned between them. In this application, unless otherwise specifically defined or limited, terms such as “installed”, “connected”, “attached”, “fixed”, and similar terms should be broadly understood. For example, they may refer to fixed connections, detachable connections, or integral connections; they may be direct connections or indirect connections through an intermediate medium, or they may refer to internal communication between two components. For those skilled in the art, the specific meaning of these terms in this application can be understood based on the context.

Unless otherwise defined, all technical and scientific terms used in this application have the meanings generally understood by those skilled in the relevant technical field.

It should be noted that the thickness, length, and width of various components shown in the accompanying drawings of the embodiments of this application, as well as the overall dimensions (thickness, length, width, etc.) of the integrated devices, are only for illustrative purposes and should not be considered as limitations of this disclosure.

The embodiments of this application will be further described with reference to the accompanying drawings.

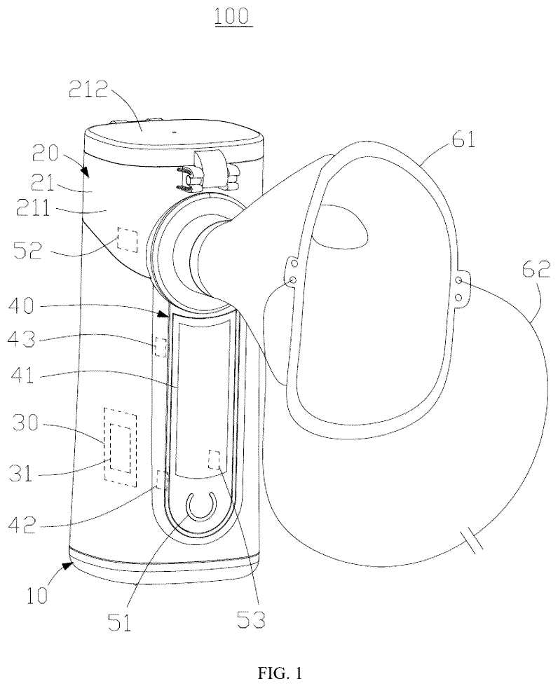

As shown in FIG. 1, an embodiment of this disclosure provides a nebulizer 100. The nebulizer 100 is a portable handheld device. When the user holds or wears the nebulizer 100, they can move freely, greatly facilitating the user's daily life or work.

In one embodiment, the nebulizer 100 includes a housing 10, an atomizing component 20, and a controller 30. The atomizing component 20 and the controller 30 are arranged within the housing 10 and electrically connected. The controller 30 can control the atomizing component 20 to work and atomize liquid in the liquid storage tank 21. In one embodiment, the controller 30 includes a circuit board 31.

In one embodiment, the atomizing component 20 includes an air compressor. When the nebulizer 100 is turned on, the air compressor starts working, compressing the air and expelling it as a high-speed airflow. The high-speed airflow creates negative pressure around a nozzle of the liquid storage tank 21, causing the medication to be drawn out of the chamber. Under the impact and shear forces of the high-speed airflow, the medication is broken down into fine mist droplets, forming nebulizer.

In another embodiment, the atomizing component 20 includes an ultrasonic transducer. The ultrasonic transducer converts electrical energy into ultrasonic vibrations, which are transmitted to the medication. When the frequency of the ultrasonic vibration matches the vibration frequency of the medication molecules, the molecules on the surface of the medication undergo repeated stretching and compression. As the vibration continues, the surface tension of the medication can no longer maintain its integrity, causing the medication to be ejected from the liquid surface and dispersed into fine mist droplets, thus forming nebulizer.

In another embodiment, the nebulizer 100 further includes a display device 40. The display device 40 includes a display screen 41, which is at least partially exposed on the housing 10 and electrically connected to the circuit board 31. The display screen 41 is configured to display parameter information of the nebulizer 100. The parameter information includes, but is not limited to, battery status, liquid volume, operating mode, nebulizer level, operation time, and at least one fault code. In this embodiment, the display screen 41 is configured to display one or more of the following parameter information: battery status, liquid volume, operating mode, nebulizer level, operation time, and fault code. This is done through text or icons, replacing traditional color-coded indicators or blinking lights, which is more user-friendly for users with color blindness or older individuals, allowing them to intuitively understand the operating status of the nebulizer 100 and reducing the difficulty of use.

In one embodiment, the display screen 41 is a light emitting diode (LED) display screen or liquid crystal display screen. Both types of display screens offer excellent presentation, providing good contrast. This not only makes it easier for users to view the parameter information on the display 41 but also helps protect the user's eyesight and reduces power consumption of the nebulizer 100, thereby extending the nebulizer 100′s battery life.

In one embodiment, the display screen 41 integrates a touch component 42, and the display screen 41 is configured for touch control. By using the touch component 42 to control the nebulizer 100, it is beneficial for the user to intuitively and flexibly control the nebulizer 100. This not only enhances the user experience but also reduces the difficulty of operation, making it easier for the elderly or children to use.

In one embodiment, the nebulizer 100 may also include a control button 51. The control button 51 is provided on the housing 10 and is electrically connected to the controller 30. The control button 51 is configured to control the working mode or nebulizer level of the atomizing component 20. By using the control button 51, the user can control the nebulizer 100, allowing for blind operation, improving convenience, and providing better reliability of the physical buttons, which helps extend lifespan of the nebulizer 100.

In one embodiment, the nebulizer 100 also equipped with the touch component 42 and the control button 51, allowing the user to choose either the touch control or the button control based on their preferences. This helps the nebulizer 100 to be compatible with users of different habits.

In one embodiment, the display screen 41 also includes an adaptive adjusting device 43. When the nebulizer 100 is in a tilted use state and the tilt angle exceeds a second threshold, the adaptive adjusting device 43 will be triggered to switch the display screen 41 from a portrait mode to a landscape mode. Similarly, when the nebulizer 100 transitions from a tilt angle above the second threshold to a tilt angle below the second threshold, the adaptive adjusting device 43 will be triggered to switch the display screen 41 from landscape mode back to portrait mode. Since the nebulizer 100 is portable, it may be tilted or placed horizontally during use. The built-in adaptive adjusting device 43 adjusts the display layout on the display screen 41 accordingly, making it easier for the user to intuitively understand the parameter information on the display screen 41, reducing the difficulty of understanding the information.

Further, the adaptive adjusting device 43 includes a gravity sensor, which is electrically connected to the controller 30. When the nebulizer 100 is flipped or tilted, the gravity sensor can convert the gravitational change into an electrical signal and send it to the circuit board 31, enabling the display to switch between portrait and landscape layouts.

In one embodiment, the second threshold for the tilt angle of the nebulizer 100 is 45°.

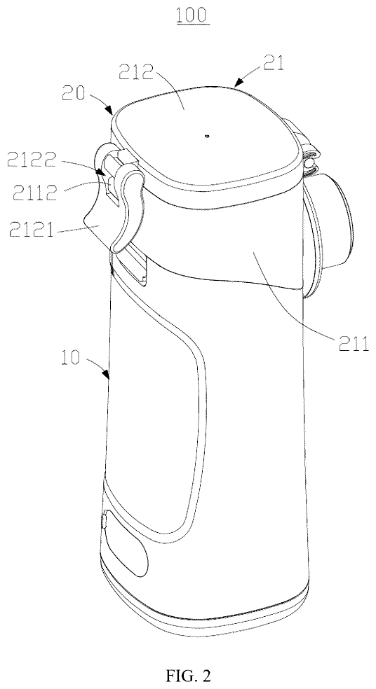

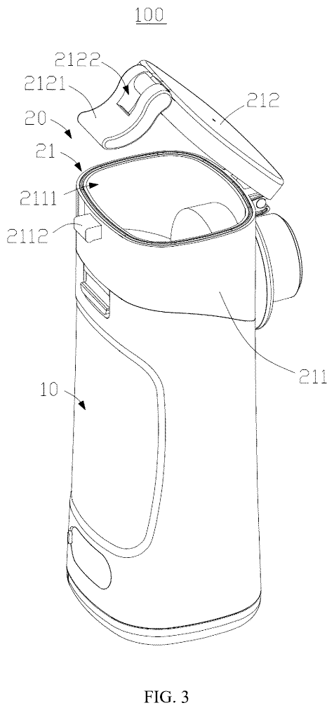

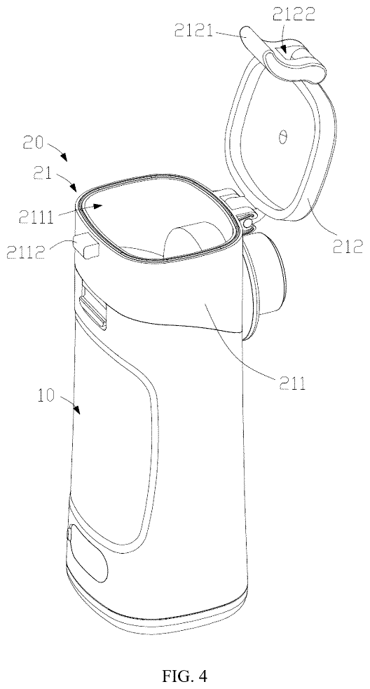

As shown in FIGS. 2 to 4, in one embodiment, the atomizing component 20 includes a liquid storage tank 21, which is used to hold liquid, such as a medicinal solution for nebulizer. In one embodiment, the liquid storage tank 21 includes a tank body 211 and a tank cover 212. The tank body 211 is connected to the housing 10, and the tank cover 212 is an openable cover on the tank body 211. When the tank cover 212 is closed on the tank body 211, the tank cover 212 and the tank body 211 form a chamber for holding liquid. When the tank cover 212 is open, the opening 2111 of the tank body 211 is exposed, allowing the user to inject liquid into the tank body 211 or pour out the liquid inside the tank.

In one embodiment, the tank cover 212 is rotatably connected to the tank body 211. By rotating the tank cover 212, it can switch between a closed and an open state, making it easy for the user to inject liquid into the tank body 211. Additionally, since the tank cover 212 and the tank body 211 are always connected, it helps prevent the tank cover 212 from being lost.

Further, one of the tank body 211 and the tank cover 212 has a connecting part 2112, and the other has a latching part 2121. When the tank cover 212 is closed on the tank body 211, the latching part 2121 can elastically snap into the connecting part 2112, securely closing the opening 2111 of the tank body 211. When it is necessary to open the tank cover 212, a force is applied away from the connecting part 2112, causing the latching part 2121 to deform elastically, thus releasing the latch between the latching part 2121 and the connecting part 2112. At this point, by rotating the tank cover 212, it can be opened. This operation is simple and convenient.

In one embodiment, the connecting part 2112 is provided on the tank body 211, and the latching part 2121 is provided on the tank cover 212. The connecting part 2112 is a protrusion located on the side surface of the tank body 211, and the latching part 2121 has a groove 2122. When the tank cover 212 is closed on the tank body 211, the protruding connecting part 2112 is inserted into the groove 2122. In one embodiment, the groove 2122 can be a recessed cavity structure with a bottom wall or a through-hole structure passing through the latching part 2121.

In one embodiment, the connecting part 2112 is arranged on the tank body 211, and the engaging part 2121 is arranged on the tank cover 212. The connecting part 2112 is a recessed portion located on the circumferential surface of the tank body 211, and the engaging part 2121 is provided with a protruding part. When the tank cover 212 is closed onto the tank body 211, the protruding part is inserted into the recessed part (not shown).

In one embodiment, the tank cover 212 is separably connected to the tank body 211 (not shown). When the tank cover 212 and the tank body 211 are separated from the closed state, the tank cover 212 can be detached from the tank body 211. Compared to a structure where the tank cover 212 and the tank body 211 cannot be separated, when the tank cover 212 is in a separated state from the tank body 211, the opening 2111 of the tank body 211 is fully exposed, which facilitates cleaning the inside of both the tank cover 212 and the tank body 211, improving their cleanliness.

In one embodiment, at least a portion of the liquid storage tank 21 is exposed on the housing 10.

The tank cover 212 is exposed on the housing 10, which facilitates the opening and closing of the tank cover 212. Furthermore, the tank cover 212 can be made of transparent material, allowing the user to observe the condition inside the liquid storage tank 21 through the tank cover 212, which helps the user intuitively understand the amount of liquid inside the storage tank 21.

In one embodiment, at least a portion of the tank body 211 is exposed on the housing 10. Furthermore, the tank cover 212 can be made of transparent material, allowing the user to observe the condition inside the liquid storage tank 21 through the sidewalls of the tank body 211, which helps the user intuitively understand the amount of liquid inside the storage tank 21.

In one embodiment, the liquid storage tank 21 is detachably connected to the housing 10. Specifically, the tank body 211 is detachably connected to the housing 10. By separating the liquid storage tank 21 from the housing 10, the tank body 211 and the tank cover 212 can be cleaned separately, which not only facilitates cleaning the liquid storage tank 21 but also prevents water from entering the interior of the housing 10 during cleaning, avoiding potential damage to other components inside the housing 10.

Referring to FIGS. 1 to 4, in one embodiment, the nebulizer 100 also includes a liquid level sensor 52, which is electrically connected to the circuit board 31. The liquid level sensor 52 is configured to detect the liquid level inside the liquid storage tank 21 and transmit the detected information in the form of an electrical signal to the circuit board 31, enabling the display screen 41 to show the current liquid level in real-time.

In one embodiment, the nebulizer 100 also includes a warning device 53, which is electrically connected to the circuit board 31. When the liquid level sensor 52 detects that the liquid level inside the liquid storage tank 21 is below a first threshold, the circuit board 31 triggers the warning device 53 to issue a warning, such as flashing an icon on the display screen 41 or emitting a warning sound, to inform the user.

In one embodiment, the first threshold is 1/10 of the total volume of the liquid storage tank 21.

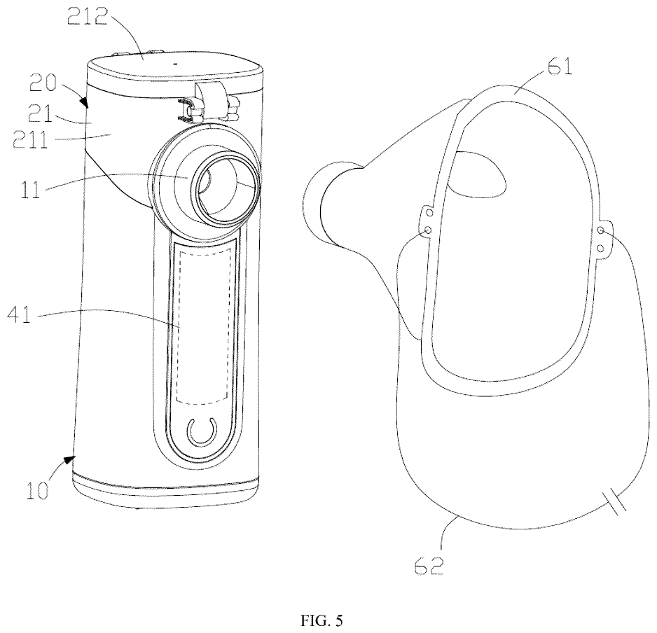

As shown in FIGS. 1 and 5, in one embodiment, the nebulizer 100 also includes a face mask 61, which is connected to the housing 10 and communicates with the atomizing component 20. The face mask 61 can be used to cover the user's mouth and nose, guiding the atomized gas output by the atomizing component 20 to the user's mouth and nose, which helps reduce the external dispersion of the atomized gas.

In one embodiment, the nebulizer 100 also includes a strap 62, with two ends of the strap 62 connected to the face mask 61, and it is configured to be worn on the user's head.

In one embodiment, the connection strength between the face mask 61 and the housing 10 satisfies the condition that when the strap 62 is worn on the user's head, the face mask 61 can support the housing 10. When the user is using or wearing the nebulizer 100, the strap 62 can be placed or wrapped around the head, with the face mask 61 covering the mouth and nose area. Through the connection between the strap 62 and the head, and the contact between the face mask 61 and the face, the face mask 61 can support the housing 10, allowing the nebulizer 100 to be fixed to the user's head without additional external support, which greatly facilitates the user's work or life while using the nebulizer 100.

In one embodiment, the surface of the housing 10 can be provided with a protruding portion 11, which extends in the direction away from the surface of the housing 10. the face mask 61 is worn over the protruding portion 11. Placing the face mask 61 over the protruding portion 11 helps increase the contact area between the face mask 61 and the housing 10, enhancing the stability of the connection between the face mask 61 and the housing 10. When the user wears the nebulizer 100 via the strap 62, the connection area between the face mask 61 and the housing 10 is less likely to deform, reducing the risk of the housing 10 separating from the face mask 61 due to gravity. Specifically, the protruding portion 11 can be ring-shaped and have a hollow structure, with the hollow structure connecting the atomizing component 20 and the face mask 61.

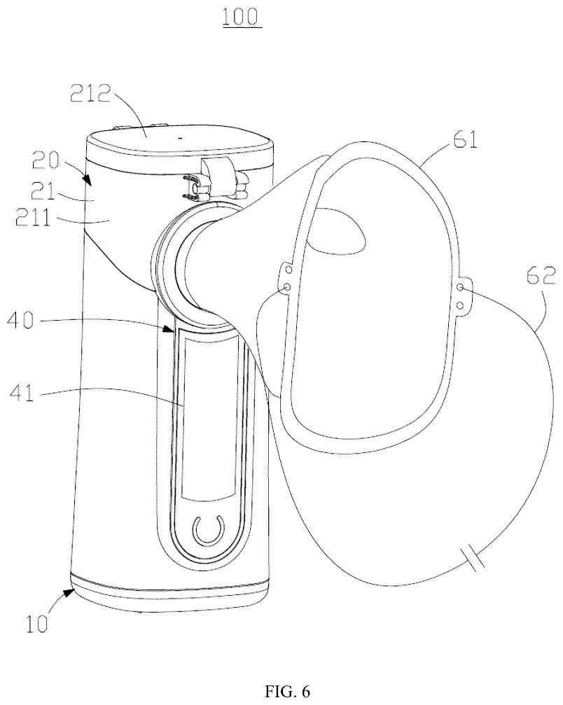

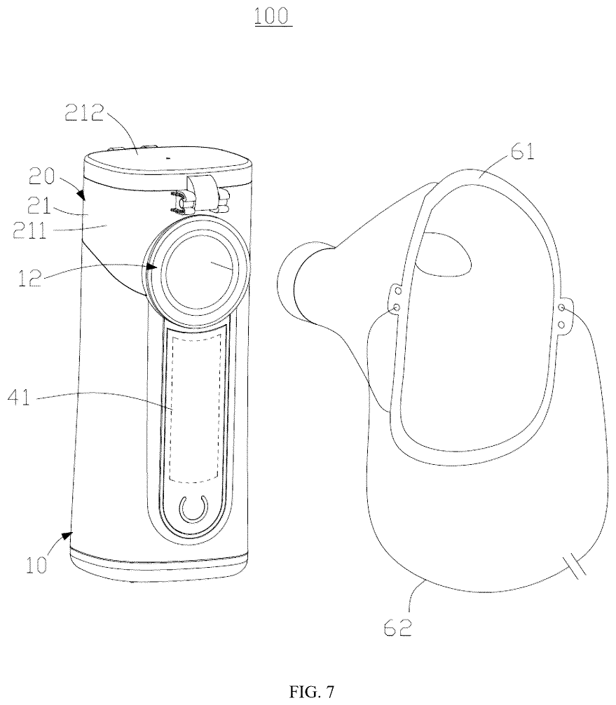

As shown in FIGS. 6 and 7, in one embodiment, the surface of the housing 10 may be provided with an annular recessed portion 12, and the face mask 61 can be inserted into the annular recessed portion 12. Inserting the face mask 61 into the annular recessed portion 12 helps to increase the contact area between the face mask 61 and the housing 10, improving the stability of the connection between the face mask 61 and the housing 10. When the user wears the nebulizer 100 using the strap 62, the connection area between the face mask 61 and the housing 10 is less likely to deform, reducing the risk of the housing 10 separating from the face mask 61 due to gravity.

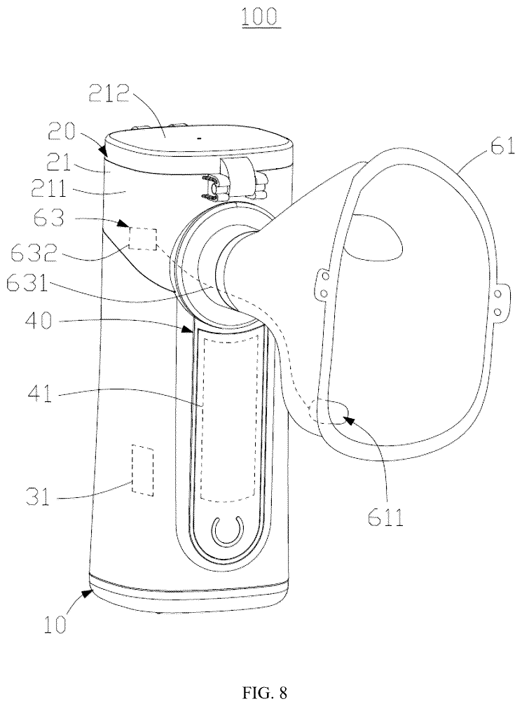

As shown in FIG. 8, in one embodiment, the nebulizer 100 further includes a reflux assembly 63, which connects the face mask 61 and the liquid storage tank 21 of the atomizing component 20. The reflux assembly 63 is configured to guide the condensed liquid inside the face mask 61 back to the liquid storage tank 21. The inventor found that during the use of the nebulizer 100, liquid condensation often occurs on the inner wall of the face mask 61, which not only leads to waste of medication but may also flow onto the user's face if the condensed droplets gather, affecting the user's experience. By providing the reflux assembly 63, the condensed liquid inside the face mask 61 can be directed back into the liquid storage tank 21, helping to reduce medication waste and preventing the condensation from negatively impacting the user's experience.

In one embodiment, the inner wall of the face mask 61 is provided with a recovery groove 611, which is configured to collect and accommodate the condensed liquid. In one embodiment, the reflux assembly 63 may include a conduit 631, which connects the recovery groove 611 and the liquid storage tank 21, allowing the liquid from the recovery groove 611 to flow back into the liquid storage tank 21 via the conduit 631. In one embodiment, the reflux assembly 63 further includes a reflux pump 632, which is connected to the conduit 631 and is used to pump the liquid from the recovery groove 611 back into the liquid storage tank 21.

It can be understood that when the reflux assembly 63 does not include the reflux pump 632, the nebulizer 100 can be inverted to allow the liquid in the recovery groove 611 to flow back into the liquid storage tank 21 under the force of gravity through the conduit 631. When the reflux assembly 63 includes the reflux pump 632, the pump can provide the necessary suction force, allowing the liquid in the recovery groove 611 to flow back into the liquid storage tank 21 through the conduit 631, even without inverting the nebulizer 100, thereby improving the user's experience.

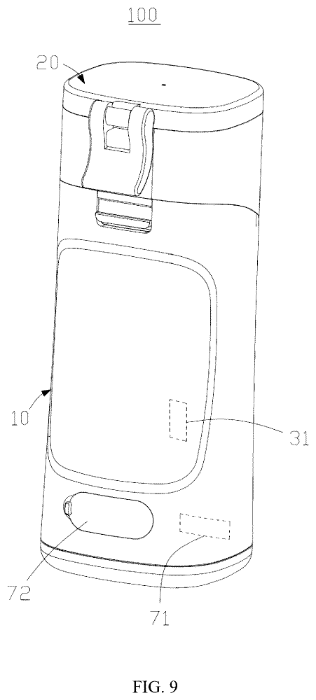

As shown in FIGS. 1 and 9, in one embodiment, the nebulizer 100 further includes a battery 71, which is located inside the housing 10 and electrically connected to the atomizing component 20, circuit board 31, display device 40, and any other electrical components. The battery 71 provides electrical power to the electrical components inside the nebulizer 100, allowing the nebulizer 100 to be used without relying on external power sources, making it easier for users to wear and move with the nebulizer 100, reducing the impact of the nebulizer process on their work or daily life.

In one embodiment, the nebulizer 100 further includes a charging interface 72, which is located on the housing 10 and electrically connected to the battery 71. The charging interface 72 is used for connecting an external charging cable to charge the battery 71.

In summary, the portable nebulizer 100 provided in this application is equipped with a display 41, which displays one or more parameters such as power information, liquid volume information, operating mode, nebulizer levels, operating time, and fault codes. This design is user-friendly for colorblind or older users, enabling them to intuitively understand the operating status of the nebulizer 100 and reducing the difficulty of using it.

The above are only some embodiments of the present disclosure, and neither the words nor the drawings can limit the protection scope of the present disclosure. Any equivalent structural transformation made by using the contents of the specification and the drawings of the present disclosure under the overall concept of the present disclosure, or directly/indirectly applied in other related technical fields are included in the protection scope of the present disclosure.

What is claimed is:

1. a nebulizer, the nebulizer is a portable handheld device, comprising a housing, an atomizing component, a controller and a display;
wherein the atomizing component is disposed in the housing, and the atomizing component is configured to atomize liquid in a liquid storage tank;
the controller is disposed in the housing, the controller is electrically connected to the atomizing component;
the display device comprises a display screen, the display screen is disposed in the housing and at least partially exposed from the housing, the display screen is electrically connected to the controller, and the display screen is configured to display parameter information of the nebulizer.

2. The nebulizer of claim 1, wherein the display screen is a light emitting diode display screen or a liquid crystal display screen.

3. The nebulizer of claim 1, wherein the display screen is integrated with a touching member, and the display screen is touch controlled.

4. The nebulizer of claim 1, wherein the nebulizer further comprises a control button, the control button is disposed in the housing and electrically connected to the controller.

5. The nebulizer of claim 1, wherein the display screen is equipped with an adaptive adjusting device;
when the nebulizer is in an inclined use state, the adaptive adjusting device is configured to switch the display screen from a portrait mode layout to a landscape mode layout.

6. The nebulizer of claim 1, wherein the nebulizer further comprises a liquid level sensor and a warning device;
wherein the liquid level sensor is configured to detect the liquid level;
when the liquid level sensor detects that the liquid level is below a first threshold, the warning device is configured to trigger the display screen to flash an icon or emit warning sound.

7. The nebulizer of claim 1, wherein the nebulizer further comprises a face mask and a reflux assembly;
wherein the face mask is connected to the housing and communicated with the atomizing component;
the reflux assembly is connected to the face mask and the liquid storage tank of the atomizing component, the reflux assembly is configured to guide the liquid condensed in the face mask back to the liquid storage tank.

8. The nebulizer of claim 7, wherein an inner wall of the face mask is provided with a recovery groove, the recovery groove is configured to hold the condensed liquid;
the reflux assembly comprise a reflux pump and a conduit, the conduit is connected to the recovery groove and the reflux pump.

9. The nebulizer of claim 1, wherein the nebulizer further comprises a face mask and a strap;
wherein the face mask is connected to the housing and communicated with the atomizing component;
two ends of the strap are connected to the face mask, and the strap is configured to be worn over a head of a user;
when the strap is worn over the head of the user, the face mask is configured to support the housing.

10. The nebulizer of claim 9, wherein a surface of the housing is provided with a protruding part, the protruding part is extended in a direction away from the surface of the housing, the face mask is worn over the protruding part.

11. The nebulizer of claim 9, wherein the surface of the housing is provided with an annular concave part, the face mask is inserted into the annular concave part.

12. The nebulizer of claim 1, wherein the atomizing component comprises a liquid storage tank, the liquid storage tank is exposed from the housing, and at least a portion of the liquid storage tank is made of a transparent material.

13. The nebulizer of claim 1, wherein the atomizing component comprises a liquid storage tank, the liquid storage tank is exposed from the housing;
the liquid storage tank comprises a tank body and a tank cover, the tank cover is rotatably connected to the tank body.

14. The nebulizer of claim 13, wherein one of the tank body and the tank cover is provided with a connecting part, and the other of the tank body and the tank cover is provided with a latching part, the latching part is detachably and elastically snap-connected to the connecting part.

15. The nebulizer of claim 1, wherein the atomizing component comprises a liquid storage tank, the liquid storage tank is exposed from the housing, and is detachably connected to the housing.
